(19) United States
(12) Patent Application Publication
(10) Pub. No.: US 2021/0369609 A1
(43) Pub. Date: Dec. 2, 2021

(54) METHOD FOR LIFTING SUBMUCOSAL OR MUCOSAL TISSUES

(71) Applicants: NATIONAL CHENG KUNG UNIVERSITY, Tainan City (TW); T-ACE MEDICAL CO., LTD., Tainan City (TW)

(72) Inventors: Xi Zhang LIN, TAINAN CITY (TW); Hui Fang LI, TAINAN CITY (TW); Chun Yao HUANG, TAINAN CITY (TW); Hung En WEI, TAINAN CITY (TW); Chih Ning WEN, TAINAN CITY (TW)

(21) Appl. No.: 16/888,049

(22) Filed: May 29, 2020

(51) Int. Cl. A61K 9/06; A61K 47/34; A61K 47/36; A61K 47/02; A61K 47/12; A61K 47/26; A61K 51/12

(52) U.S. Cl. CPC A61K 9/06; A61K 47/34; A61K 47/36; A61K 51/1213; A61K 47/12; A61K 47/26; A61K 47/02

(57) ABSTRACT

The present disclosure provides a method for lifting submucosal or mucosal tissues in a patient, comprising administrating to the tissues an effective amount of a thermosensitive gel composition comprising Poloxamer-407. A method for performing an endoscopic procedure, a method for submucosal or mucosal resectioning, a method for performing an endoscopic ultrasound procedure, and a method for performing a polyp removal are also provided.

### BACKGROUND

### 1. Field of the Disclosure

The present disclosure relates generally to methods for lifting submucosal or mucosal tissues during medical procedures.

### 2. Description of the Related Art

A conventional method for lifting submucosal or mucosal tissues during medical procedure typically involves using a composition comprising a physiological saline, glycerol, hyaluronic acid, glucose solution, or hydroxypropyl methylcellulose. However, typically, the composition that can be easily injected can also easily flow away from the tissue after the injection. On the other hand, the compositions which maintains tissue localization is usually not easily injected. Moreover, the composition with high fluidity usually fails to maintain the lifting property for a long time and further causes damages to the deeper tissue during operating resection procedures.

Copolymer of ethylene oxide and propylene oxide blocks is widely used in in situ-forming systems. Poloxamer-407 exhibits good thermosensitive properties, which has been applied in drug formulation.

However, temperature control of the injectable mixture is quite critical when utilizing the thermosensitive Poloxamer-407. For example, when Poloxamer-407 is charged in a fine syringe, it is easy to turn into a gel form from an aqueous form, causing pipeline blockage. For that reason, Poloxamer-407 must be kept in a low temperature before use. Therefore, when Poloxamer-407 enters the tissue, the temperature usually remains below the gel-forming temperature, and the elevation rate of temperature varies depending on the individual and the environment. Furthermore, the thermosensitive property of Poloxamer-407 also relies on its concentration. Since Poloxamer-407 remains in the aqueous form when entering the tissue, it tends to be mixed with blood or tissue fluid, being diluted or washed away, and then leading the thermosensitive property change.

Accordingly, there is a strong need for the development of a method for lifting submucosal or mucosal tissues during medical procedures.

### SUMMARY

In some embodiments, the present disclosure provides a method for lifting submucosal or mucosal tissues in a patient, comprising administrating to the tissues an effective amount of a thermosensitive gel composition comprising Poloxamer-407.

In some embodiments, the present disclosure provides a method for performing a gastrointestinal endoscopic procedure comprising the method as mentioned above.

In some embodiments, the present disclosure provides a method for submucosal or mucosal resectioning comprising the method as mentioned above.

In some embodiments, the present disclosure provides a method for performing an endoscopic ultrasound procedure comprising the method as mentioned above.

In some embodiments, the present disclosure provides a method for performing a polyp removal comprising the method as mentioned above.

### BRIEF DESCRIPTION OF THE DRAWINGS

FIG. 1 shows the results of lifting pig intestine by using the exemplary thermosensitive gel composition.

FIG. 2 shows the results of lifting pig intestine by using the exemplary thermosensitive gel composition.

FIGS. 3 (A) and (B) show the results of lifting pig intestine by using the exemplary thermosensitive gel composition. (A) Left: After one hour, the lifting effect of the first T-AceGel composition was from 1.58 cm to 1.28 cm, with 0.3 cm decreasing (19%). Right: After one hour, the lifting effect of water was from 0.83 cm to 0.27 cm, with 0.56 cm decreasing (67%). (B) Left: After one hour, the lifting effect of the first T-AceGel composition was from 1.33 cm to 1.18 cm, with 0.15 cm decreasing (11%). Right: After one hour, the lifting effect of 10% glycerol was from 1.43 cm to 0.89 cm, with 0.54 cm decreasing (38%).

FIGS. 4 (A) and (B) show the thermosensitive property of the exemplary thermosensitive gel composition. (B) Into a vial, 3 c.c. of the first T-AceGel composition or 3 c.c. of the second T-AceGel composition was injected.

FIGS. 5 (A) to (I) show the result of disintegration of the exemplary thermosensitive gel composition.

### DETAILED DESCRIPTION

The present disclosure provides a method for lifting submucosal or mucosal tissues in a patient, comprising administrating to the tissues an effective amount of a thermosensitive gel composition comprising Poloxamer-407. As used herein, subject/patient can include mammals, such as, for example, humans. In another embodiment, a pharmaceutically acceptable formulation suitably formulated for said method above is also provided.

Preferably, the thermosensitive gel composition further comprises Poloxamer-188.

Preferably, the thermosensitive gel composition further comprises an excipient.

Preferably, the excipient is selected from the group consisting of sodium chloride, calcium chloride, calcium lactate, monosaccharide, and polysaccharide.

Preferably, the thermosensitive gel composition further comprises a dye. More preferably, the dye is Brilliant blue FCF or indiocarmine.

Preferably, the thermosensitive gel composition further comprises alginate. While not limited by theory, in one embodiment, alginate is able to conjugate with the calcium ion to form a gel.

Preferably, the thermosensitive gel composition further comprises a drug or radioactive compound.

Preferably, the content of Poloxamer-407 is about 13% to about 20% (e.g. 14%, 15%, 16%, 17%, 18%, 19%) by weight of the thermosensitive gel composition; more preferably, about 16% to about 18%.

Preferably, the content of Poloxamer-188 is about 0.5% to about 3% (e.g. 1.0%, 1.5%, 2%, 2.5%) by weight of the thermosensitive gel composition; more preferably, about 1% to about 3%.

Preferably, the content of the excipient is about 0.05% to about 6% by weight of the thermosensitive gel composition. In a more preferred embodiment, the excipient is sodium chloride, and the content of sodium chloride is about 0.05% to about 5% by weight of the thermosensitive gel composition; more preferably, about 0.1% to about 0.3%. In a more preferred embodiment, the excipient is calcium chloride, and the content of calcium chloride is about 0.05% to about 5% by weight of the thermosensitive gel composition; more preferably, about 0.1% to about 0.3%. In a more preferred embodiment, the excipient is calcium lactate, and the content of calcium lactate is about 0.1% to about 1% by weight of the thermosensitive gel composition; more preferably, about 0.4% to about 0.5%. In a more preferred embodiment, the excipient is sucrose, and the content of sucrose is about 1% to about 5% by weight of the thermosensitive gel composition; more preferably, about 1% to about 3%.

Preferably, the content of the dye is about 0.01% to about 0.025% by weight of the thermosensitive gel composition.

Preferably, the content of alginate is about 0.1% to about 1.0% by weight of the thermosensitive gel composition.

Preferably, the method according to the instant disclosure comprises administrating to the tissues an effective amount of a first thermosensitive gel composition and then administrating to the tissues an effective amount of a second thermosensitive gel composition; wherein the first thermosensitive gel composition comprises Poloxamer-407, and the second thermosensitive gel composition comprises Poloxamer-407 and alginate.

Preferably, the first thermosensitive gel composition further comprises Poloxamer-188.

Preferably, the first thermosensitive gel composition further comprises an excipient.

Preferably, wherein the first thermosensitive gel composition further comprises a dye.

In a more preferred embodiment of the disclosure, the content of Poloxamer-407 is about 13% to about 16% by weight of the second thermosensitive gel composition; and the content of alginate is about 0.1% to about 1% by weight of the second thermosensitive gel composition.

The present disclosure provides a method for performing an endoscopic procedure comprising the method as mentioned above. Examples of the endoscopic procedure include but are not limited to lesion marking, repeated submucosal injection, mucosal dissection, repeated submucosal exfoliation, and complete lesion removal.

The present disclosure provides a method for submucosal or mucosal resectioning comprising the method as mentioned above.

The present disclosure provides a method for performing an endoscopic ultrasound procedure comprising the method as mentioned above.

In some embodiments of the present disclosure, it is to provide a method for performing a polyp removal comprising the method as mentioned above.

The following examples are given for the purpose of illustration only and are not intended to limit the scope of the present invention.

### EXAMPLE 1

First Thermosensitive Gel (Thermosensitive-AceGel, T-AceGel) Composition

The formations of the first T-AceGel composition are shown in Table 1.

TABLE 1

| | 1. | 2. | 2. |
|---|---|---|---|
| Component | Range % | Range % | Range % |
| Poloxamer-407 | 14~20 | 18~20 | 14~16 |
| Poloxamer-188 | 0 | 1~3 | 0.5~3 |
| Sodium chloride | 0.1~0.3 | 0.1~0.5 | 0.05~0.2 |
| Calcium chloride | 0.1~0.4 | 0.1~0.3 | 0.05~0.2 |
| Calcium Lactate | 0.1~0.5 | 0 | 0.4~1 |
| Sucrose | 1~3 | 1~5 | 1~3 |
| Brilliant blue FCF | 0.01~0.025 | 0.01~0.025 | 0.01~0.025 |
| (with or without) | | | |

Poloxamer-407 was stirred at 4° C. with 350-450 rpm, and other components except dye were poured into Poloxamer-407 slowly for 2 to 6 hours. After all solids were dissolved, the dye was added and stirred for 10 minutes.

### EXAMPLE 2

Second T-AceGel Composition

The formations of the second T-AceGel composition are shown in Table 2.

TABLE 2

| | 1. |
|---|---|
| Component | Range % |
| Poloxamer-407 | 13-16 |
| Alginate | 0.1~1.0 |

Poloxamer-407 was stirred at 4° C. with 350-450 rpm, and then alginate was added into Poloxamer-407 slowly for 2 to 4 hours.

### EXAMPLE 3

Method for Lifting Pig Intestine

The exemplary pig intestine was cut properly and put on a hot plate to keep the temperature at 37° C. An external thermometer was linked with the pig's intestine to monitor the intestine temperature.

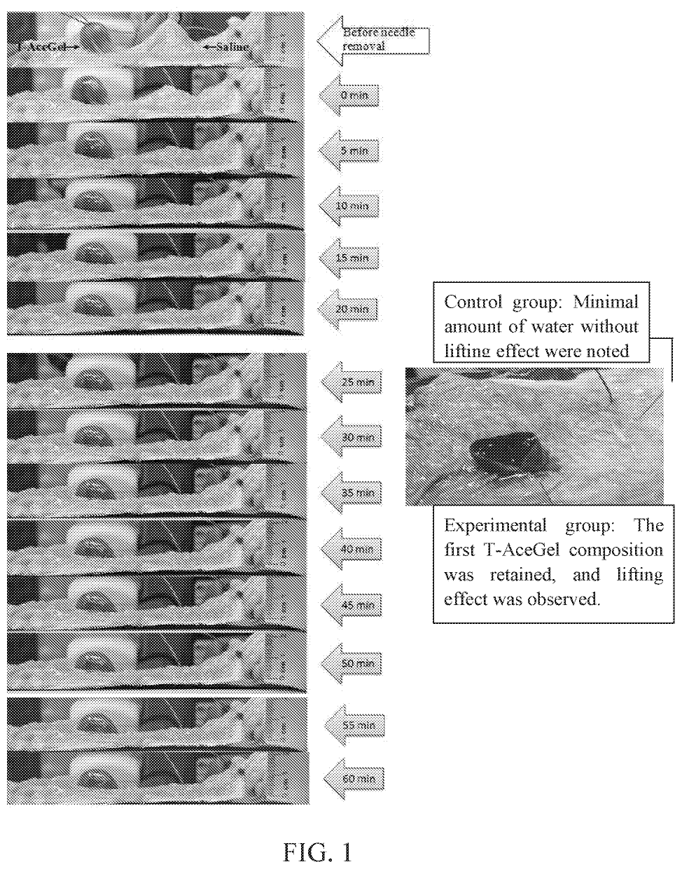

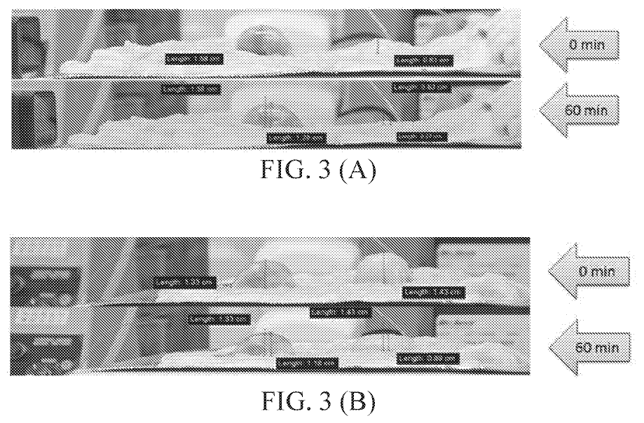

Five-c.c. of the first T-AceGel composition (left) or water for injection (right; no staining), respectively was injected to the pig intestine, and the effect of lifting was observed by taking pictures every 5 minutes. The results are shown in FIGS. 1 and 3 (A) and (B). The lifting effect of the first T-AceGel composition after 60 minutes is from 1.58 cm to 1.28 cm, with 19% decreasing. The lifting effect of water after 60 minutes is from 0.83 cm to 0.27 cm, with 67% decreasing.

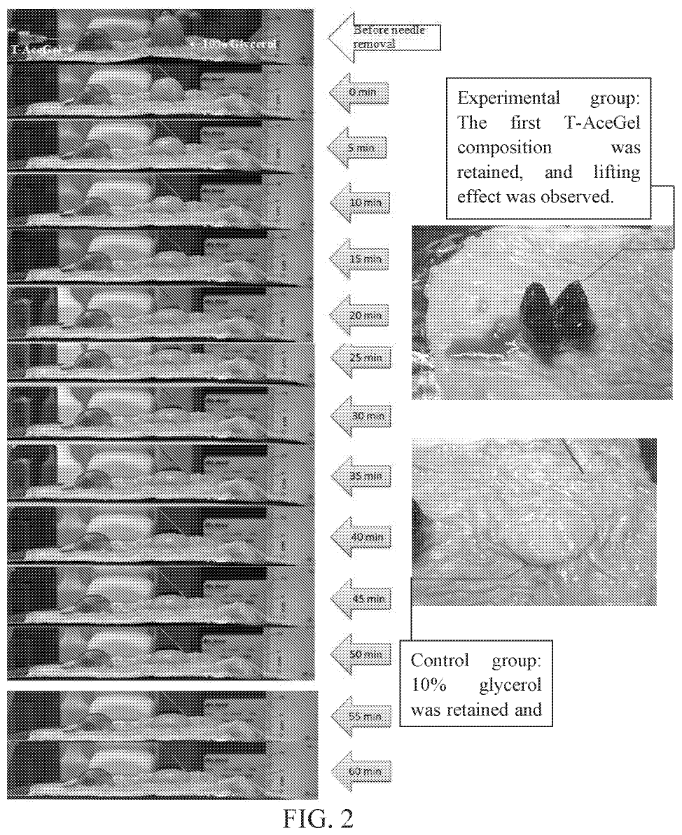

Five-c.c. of the first T-AceGel composition (left) or 10% glycerol for injection (right; no staining), respectively was injected to the pig intestine, and the effect of lifting was observed by taking pictures every 5 minutes. The results are shown in FIGS. 2 and 3 (A) and (B). The lifting effect of the first T-AceGel composition after 60 minutes is from 1.33 cm to 1.18 cm, with 11% decreasing. The lifting effect of 10% glycerol after 60 minutes is from 1.43 cm to 0.89 cm, with 38% decreasing.

### EXAMPLE 4

Thermosensitive Property

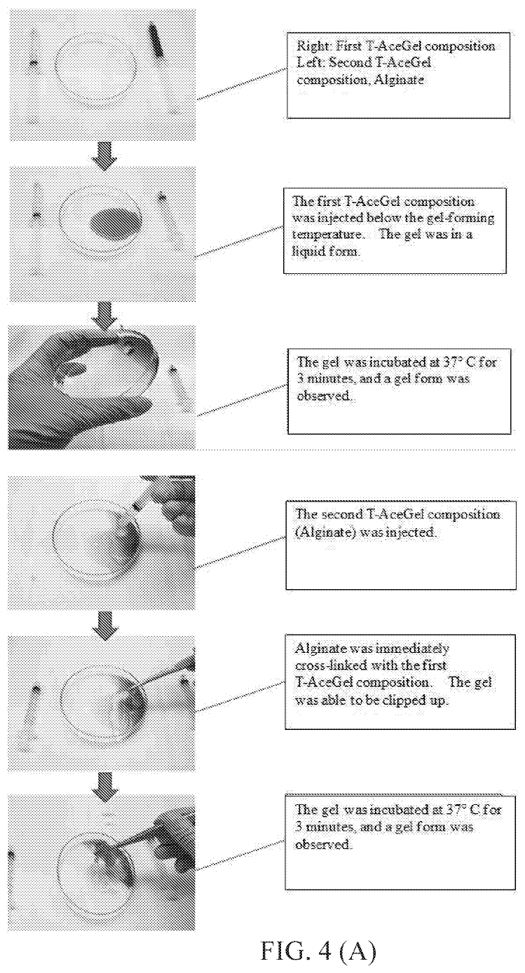

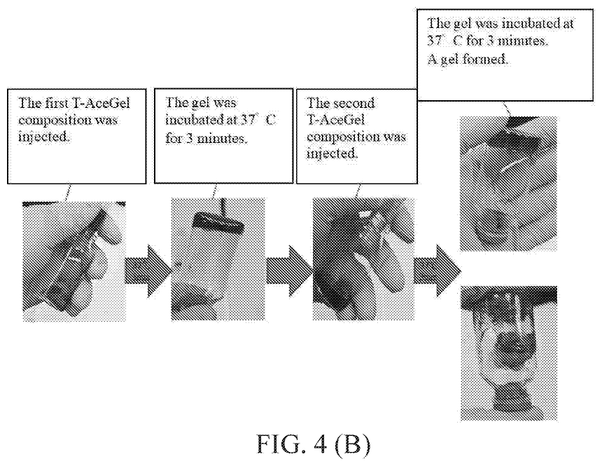

Three-c.c. of the first T-AceGel composition or the second T-AceGel composition, respectively was charged in a syringe. The first T-AceGel composition was injected to a glass plate and incubated at 37° C. for 3 minutes, and then the second T-AceGel composition was injected and further incubated at 37° C. for 3 minutes as shown in FIGS. 4 (A) and (B).

### EXAMPLE 5

Disintegration

Twenty-five-mL of 1% agarose (w/w) was placed into a glass plates. After cooling, a circle hole with a diameter of 2 cm in the middle position was dig and incubated at 37° C. An external thermometer was applied to monitor the temperature.

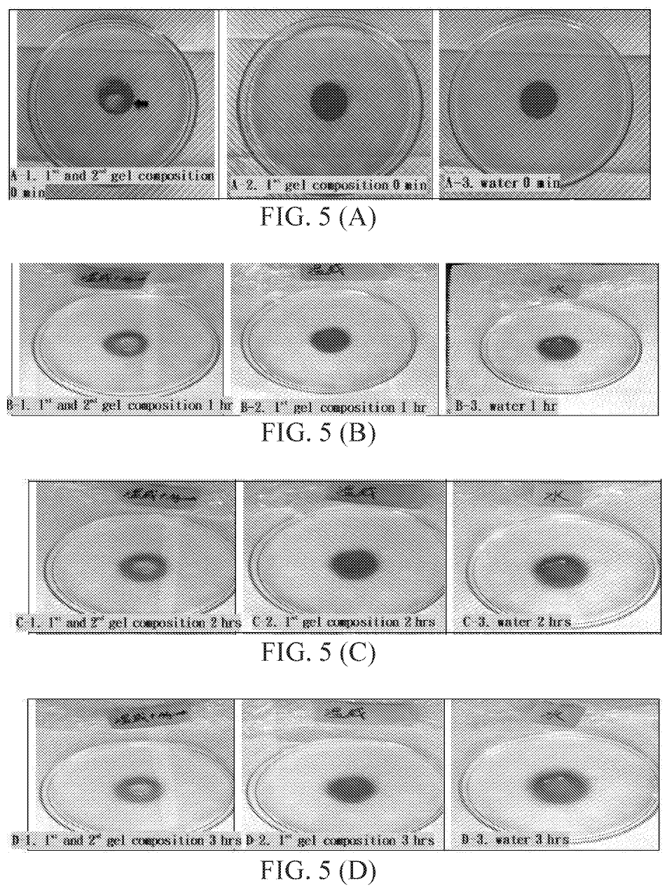

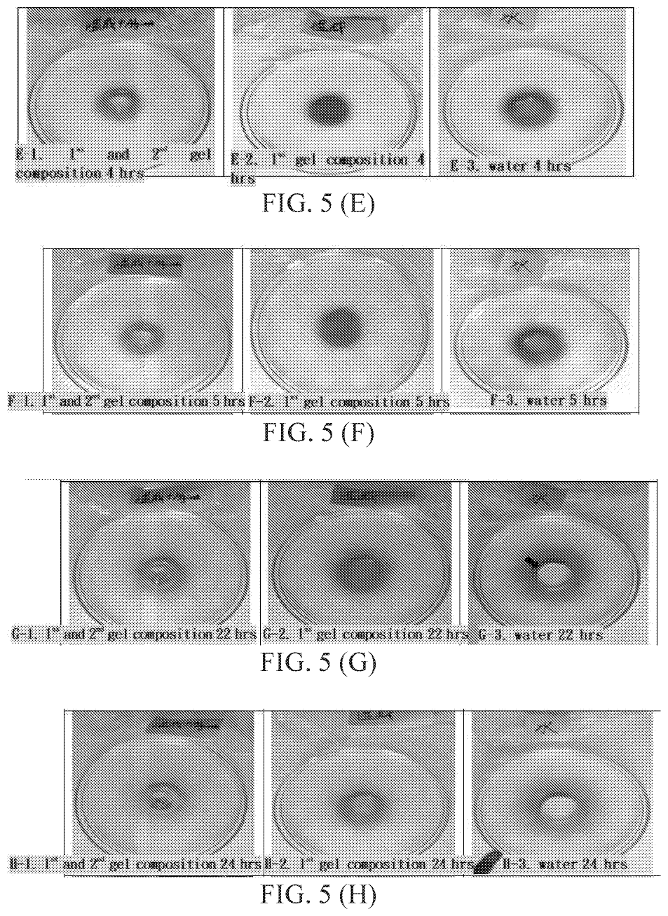

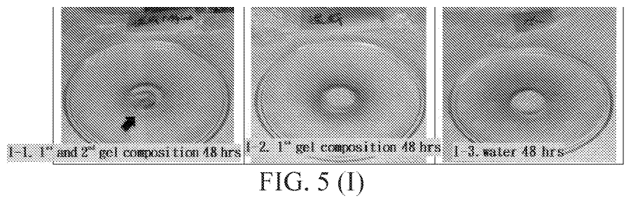

Four degree C. of 1 c.c. of water for injection, 1 c.c. of the first T-AceGel composition, or 0.5 c.c. of the first T-AceGel composition+0.5 c.c. of the second T-AceGel composition, respectively was injected to the hole. The result of disintegration was assayed at 37° C. as shown in FIGS. 5 (A) to (I).

As shown in FIGS. 5 (A) to (I), the gel is well form immediately in the first T-AceGel composition+the second T-AceGel composition group (marked by the arrow in A-1), while the first T-AceGel composition does not reach the gel-forming temperature as liquid (A-2). After 1 hour, the first T-AceGel composition is not completely gelled, and only becomes thicker. It might be because the agarose affects the concentration (B-1, B-2), and the three samples have a little outward penetration (B-1, B-2, B-3). As time goes by, water penetrates the fastest and has the widest range (indicated by the arrow in G-1). The first T-AceGel composition is still not completely gelled, and only becomes thicker (C-2 to H-2 chart). However, the range of penetration is slower and narrower than water (I-1, 1-3 chart). The first T-AceGel composition+the second T-AceGel composition group is still not completely gelled, and only becomes thicker (C-1 to H-1 chart). However, the range of penetration is not significantly different from the first T-AceGel composition (I-1, 1-2). However, the range of penetration is slower and narrower than water (I-1, 1-3) than water (I-1, 1-2). The first T-AceGel composition+the second T-AceGel composition group can still stay (I-1, black arrow mark).

While the present disclosure has been described and illustrated with reference to specific embodiments thereof, these descriptions and illustrations are not limiting. It should be understood by those skilled in the art that various changes may be made and equivalents may be substituted without departing from the true spirit and scope of the present disclosure as defined by the appended claims. The illustrations may not be necessarily drawn to scale. There may be distinctions between the artistic renditions in the present disclosure and the actual apparatus due to manufacturing processes and tolerances. There may be other embodiments of the present disclosure which are not specifically illustrated. The specification and drawings are to be regarded as illustrative rather than restrictive. Modifications may be made to adapt a particular situation, material, composition of matter, method, or process to the objective, spirit and scope of the present disclosure. All such modifications are intended to be within the scope of the claims appended hereto. While the methods disclosed herein have been described with reference to particular operations performed in a particular order, it will be understood that these operations may be combined, sub-divided, or re-ordered to form an equivalent method without departing from the teachings of the present disclosure. Accordingly, unless specifically indicated herein, the order and grouping of the operations are not limitations of the present disclosure.

What is claimed is:

1. A method for lifting submucosal or mucosal tissues in a patient, comprising administrating to the tissues an effective amount of a thermosensitive gel composition comprising Poloxamer-407.

2. The method according to claim 1, wherein the thermosensitive gel composition further comprises Poloxamer-188.

3. The method according to claim 1, wherein the thermosensitive gel composition further comprises an excipient.

4. The method according to claim 3, wherein the excipient is selected from the group consisting of sodium chloride, calcium chloride, calcium lactate, monosaccharide, and polysaccharide.

5. The method according to claim 1, wherein the thermosensitive gel composition further comprises a dye.

6. The method according to claim 1, wherein the thermosensitive gel composition further comprises alginate.

7. The method according to claim 1, wherein the thermosensitive gel composition further comprises a drug or radioactive compound.

8. The method according to claim 1, wherein the content of Poloxamer-407 is about 13% to about 20% by weight of the thermosensitive gel composition.

9. The method according to claim 2, wherein the content of Poloxamer-188 is about 0.5% to about 3% by weight of the thermosensitive gel composition.

10. The method according to claim 3, wherein the content of the excipient is about 0.05% to about 6% by weight of the thermosensitive gel composition.

11. The method according to claim 5, wherein the content of the dye is about 0.01% to about 0.025% by weight of the thermosensitive gel composition.

12. The method according to claim 6, wherein the content of alginate is about 0.1% to about 1.0% by weight of the thermosensitive gel composition.

13. The method according to claim 1, which comprises administrating to the tissues an effective amount of a first thermosensitive gel composition and then administrating to the tissues an effective amount of a second thermosensitive gel composition; wherein the first thermosensitive gel composition comprises Poloxamer-407, and the second thermosensitive gel composition comprises Poloxamer-407 and alginate.

14. The method according to claim 13, wherein the first thermosensitive gel composition further comprises Poloxamer-188.

15. The method according to claim 13, wherein the first thermosensitive gel composition further comprises an excipient.

16. The method according to claim 15, wherein the excipient is selected from the group consisting of sodium chloride, calcium chloride, calcium lactate, monosaccharide, and polysaccharide.

17. The method according to claim 13, wherein the first thermosensitive gel composition further comprises a dye.

18. A method for performing an endoscopic procedure comprising the method according to claim 1.

19. A method for submucosal or mucosal resectioning comprising the method according to claim 1.

20. A method for performing an endoscopic ultrasound procedure comprising the method according to claim 1.

21. A method for performing a polyp removal comprising the method according to claim 1.
